(19) United States
(12) Patent Application Publication
(10) Pub. No.: US 2026/0204398 A1
(43) Pub. Date: Jul. 16, 2026

(54) INTEGRATED HEALTHCARE SYSTEM AND ASSOCIATED METHOD

(71) Applicant: Global Health Mam Ltd, London (GB)

(72) Inventor: Muhammed Abdul MALIK, London (GB)

(73) Assignee: Global Health Mam Ltd, London (GB)

(21) Appl. No.: 19/148,910

(22) PCT Filed: Jan. 18, 2024
(86) PCT No.: PCT/GB2024/050140

§ 371 Date: Jul. 17, 2025

(30) Foreign Application Priority Data
Jan. 19, 2023 (GB) 2300820.4

(51) Int. Cl. G16H 40/20; G16H 50/30

(52) U.S. Cl. CPC G16H 40/20; G16H 50/30

(57) ABSTRACT

An integrated healthcare system including at least one general practitioner, at least one optician, at least one dentist, at least one ear, nose and throat specialist, and at least one diagnostics centre. A healthcare-system on-boarding method of identifying a healthcare package for a patient to an integrated healthcare system. The method comprises assessing a patient's geographical history data and application data (S101, S102) via a patient electronic device to determine whether a threshold level is reached (S103). Proposed package data is then displayed to a prospective patient based on whether a threshold level is reached (S107).

The present invention relates to an integrated healthcare system and an on-boarding method of identifying a healthcare package for a patient for the or another integrated healthcare system.

Conventional healthcare services are expensive to operate. This is particularly the case due to inefficiencies in the healthcare system.

Additional costs may be caused by patient deterioration during long waiting times for further treatment. For example, a patient may wait a long time between being referred by a general practitioner or a primary care physician for additional treatment, and that additional treatment taking place. This may be partly due to the additional treatment taking place in a different location on a different day. Since the patient has deteriorated during the waiting time, more expensive and intrusive medical intervention may be required.

Furthermore, patients may miss follow-up appointments, and thereby waste the time of healthcare professionals. As such, more healthcare professionals are needed to be employed to cover wasted time caused by non-attendance of appointments, increasing the cost of the healthcare service.

Additionally, different parts of the healthcare services may be separate from each other, and thereby have individual administrative systems, rather than sharing a single administrative system. The requirement for separate administrative systems may incur additional costs.

Therefore, it can be seen that for any healthcare service, whatever funding technique is used, cost savings can be made via a reformed healthcare system.

In addition, healthcare services may be inaccessible for many individuals in low-income households in least developed countries. This is since there may be no national or general healthcare system, and patients are required to make payment for specific services. Such payments may be relatively large, and such individuals may not be able to afford such a large amount at the time when payment is required.

It would be desirable to optimise the level of payments required to be made by such individuals.

The present invention seeks to provide a solution to these problems.

According to a first aspect of the present invention, there is provided a healthcare-system on-boarding method of identifying a healthcare package for a patient to an integrated healthcare system by determining a general physical state of the patient, the method comprising: at an electronic device with a processor, memory, and a display screen: assigning a first data value to geographical history data in said memory; assigning a second data value to application data installed on said electronic device; based on the first and second data values, determining whether a threshold level is reached; if the threshold level is not reached, applying a first user health attribute; if the threshold level is reached or exceeded, applying a second user health attribute; outputting the determined first or second user health attribute to an integrated-healthcare-system server; receiving from the integrated-healthcare-system server a response based at least in part on the outputted determined first or second user health attribute which comprises proposed package data; and displaying the proposed package data on the display screen of the said electronic device.

As such, this may be considered to be a method of remotely determining a general physical state of a prospective patient when registering or onboarding them. This may in effect provide a risk assessment to assess the level of payments which they may need to pay. The amount of geographical movement a prospective patient makes can provide an indication of their exercise regimen and therefore likely physical state or general health. Similarly, the types or duration of use of various applications (apps) on their electronic device can provide an indication of how much they utilise their electronic device, and therefore provide an indication of their likely physical state or general health.

Since this data is being obtained directly from the electronic device, a patient is not required to take time answering a questionnaire, and a patient cannot attempt to deceive the assessment by lying in a questionnaire.

The general physical state is determined by real world data which may be measured by the electronic device, and as such represents an improvement of the technical task of determining a general physical state of a prospective patient.

Patients are able to access treatment via making payment according to their likely required use of the integrated healthcare system. This may make the healthcare system more accessible for certain patients.

However, it will be appreciated that such a method may be supplemented by the use of questionnaires or physical assessments of a patient.

Preferably, the geographical history data may be at least in part based on a duration or distance of movement of the electronic device in a time period, wherein the movement has a speed of less than 9 metres per second [20.1 miles per hour]. Such a limitation can prevent or limit geographical movement by motorised transport being accounted for in the assessment, whilst avoiding most movement via cycling being excluded.

Advantageously, whether a threshold level is reached based on the geographical history data may be at least in part based on a predominant location of the device. Beneficially, the memory or the integrated-healthcare-system server may have location risk-factor correspondence data including a plurality of geographic locations and at least one health risk-factor associated with each location, whether a threshold level is reached based on the geographical history data is at least in part based on the corresponding health risk-factor associated with the location. As such, various risk factors based on a person's location can be considered. For example, if a person spends a large amount of time in an urban environment, they may be at risk from the health effects of air pollution. Thus, a risk may therefore be accounted for in the on-boarding process.

In a preferable embodiment, the application data may be at least in part based on determining the number of applications installed on said electronic device in a first class and second class.

Preferably, the application data may be at least in part based on determining a duration of use of applications installed on said electronic device within a first class and second class

Optionally, the first class may be lifestyle, and the second class may be gaming or social media. Heavy use of lifestyle applications, such as exercise-related applications or mental-health-related applications, can be suggestive of a prospective patient's good health. Contrastingly, heavy use of gaming or social media applications can be suggestive of a prospective patient's ill health.

Preferably, the electronic device may include a global positioning system, cellular location system and/or an assisted global positioning system which may generate or measure the geographical history data.

According to a second aspect of the invention, there is provided an integrated healthcare system using a healthcare-system on-boarding method according to a first aspect of the invention, the integrated healthcare system comprising: a building structure at a single site; a plurality of departments within the building structure, the plurality of departments including at least one general practitioner; at least one optician; at least one dentist; at least one ear, nose and throat specialist; at least one diagnostics centre, and at least one pharmacy; an integrated-healthcare-system server in communication with the said electronic device; a patient registration system on the said integrated-healthcare-system server, the patient registration system including a plurality of different patient packages; one said patient package being selectable and sendable to the said electronic device based at least in part on the outputted determined first or second user health attribute.

Since all the departments are in the same building structure, the system has a greater efficiency than if the departments were separate. For example, the same administrative system can be used, saving the cost of duplicate administrative systems for the different departments. Additionally, a patient can more easily be referred between departments, and can attend appointments in different departments on the same day. This reduces the risk of a patient cancelling a referred appointment, and thereby reduces the amount of medical-professional wasted time. Furthermore, since a referred appointment can be immediately after an initial appointment, the patient is less likely to deteriorate, and so less complex and less expensive treatment procedures may necessary.

Preferably, the integrated-healthcare-system server may include a booking system for patients to book appointments with the at least one general practitioner; the at least one optician; the at least one dentist; the at least one ear, nose and throat specialist; and/or the at least one diagnostics centre.

Beneficially, the booking system may be for patients to book multiple appointments with the at least one general practitioner; the at least one optician; the at least one dentist; the at least one ear, nose and throat specialist; and/or the at least one diagnostics centre.

Advantageously, the integrated-healthcare-system server may be configured to send a reminder to the said electronic device for an appointment. As such, the patient is less likely to miss an appointment and so less likely to waste the time of a medical professional.

Additionally, the integrated healthcare system may further comprise at least one department video camera for imaging the department of at least one general practitioner; at least one optician; at least one dentist; at least one ear, nose and throat specialist; and/or at least one diagnostics centre, the electronic device having a video display, the integrated-healthcare-system server configured to communicate with the electronic device so that the video display of the electronic device displays a video feed of the department video camera.

Additionally, the integrated healthcare system may further comprise at least one department video display at the at least one general practitioner; at least one optician; at least one dentist; at least one ear, nose and throat specialist; and/or at least one diagnostics centre, the electronic device having a camera for imaging the patient, the integrated-healthcare-system server configured to communicate with the electronic device so that the department video display displays a video feed of the camera of the electronic device.

As such, the patient has the option of carrying out a video appointment with the departments. Additionally or alternatively, a patient may be able to physically see a queue for the departments, and therefore may decide on whether the attend the health facility. However, it will be appreciated that other patients in the queue may not wish to be seen by other patients. In this instance, to protect patient privacy, the camera may be positioned at an angle from which it is difficult to see the patients'faces, but from which the number of patients can still be counted. For example, the camera may be positioned directly above the patients, so that only the tops of their heads are visible. Additionally or alternatively, if a person's face is in view of the camera, artificial intelligence or a processor may be configured to detect the presence of the face, and automatically blur the face in the video feed, to protect patient privacy.

Additionally or alternatively, there may be displayed a numerical indication on a patient's electronic device of the number of patients in the queue. As such, a reception of the integrated healthcare system may monitor and record the number of patients in a queue, so that this can be transmitted to the relevant electronic devices.

Preferably, the patient package may include access to the general practitioner over a duration for a plurality of appointments at a fixed cost.

Advantageously, the package may provide the option of an additional payment for access to the optician, the ear, nose and throat specialist and/or at least one diagnostics centre over a duration for a plurality of appointments at a fixed cost.

As such, a patient, particularly a low-income patient, may be more easily able to budget for their healthcare requirements.

Beneficially, the duration may be a year, access may be for a predetermined number of appointments dependent on the selected package, and the fixed cost may be paid monthly.

In a preferable embodiment, the patient package may provide a discount on further medical products and/or services. These may be dispensed from the pharmacy.

Preferably, the further medical products and/or services may include medicines, consultations and/or treatments.

The invention will now be more particularly described, by way of example only, with reference to the accompanying drawings, in which:

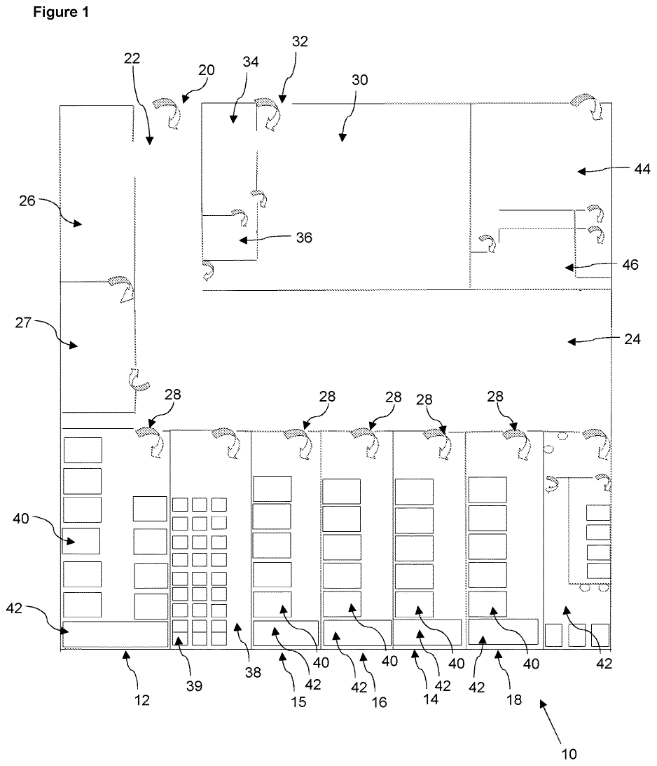

FIG. 1 shows a representation of a first embodiment of a building structure at a single site having a plurality of departments of an integrated healthcare system in accordance with a second aspect of the invention;

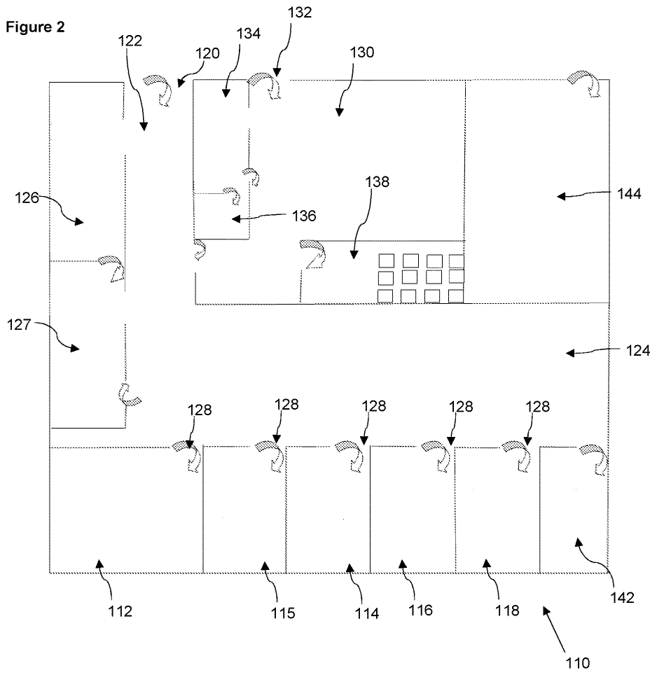

FIG. 2 shows a representation of a second embodiment of a building structure at a single site having a plurality of departments of an integrated healthcare system in accordance with a second aspect of the invention; and

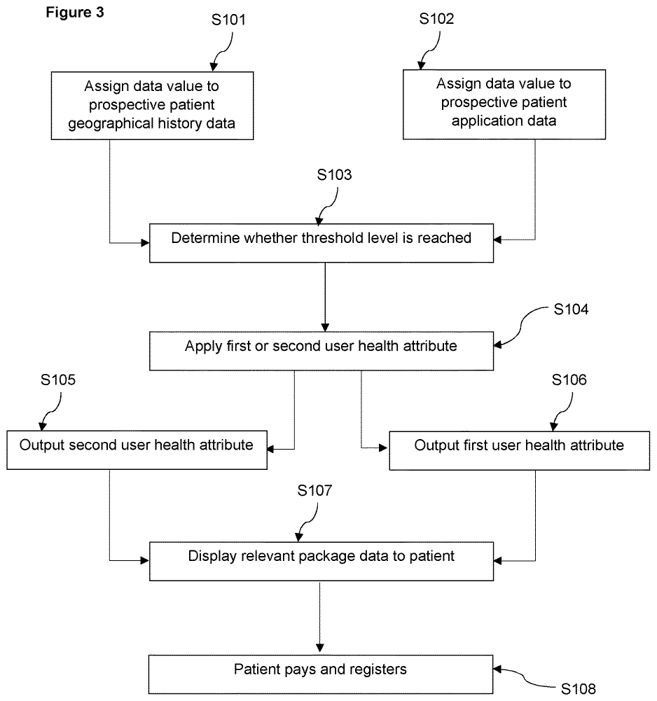

FIG. 3 shows a representation of a healthcare-system on-boarding method in accordance with a first aspect of the invention.

Referring firstly to FIG. 1, there is shown a first embodiment of an integrated healthcare facility of an integrated healthcare system. The facility includes a building structure 10 at a single site having a plurality of departments. The building structure 10 is a healthcare building structure 10, and the departments are healthcare departments.

The healthcare departments include at least one general practitioner 12, at least one optician 14, at least one dentist 15, at least one ear, nose and throat (ENT) specialist 16, and at least one diagnostics centre 18.

There is preferably a main access 20 to the building structure 10 which leads to a single reception area 22 and a single waiting area 24 for all the departments. The reception area 22 includes a reception staff area 26 and a management room 27. At the waiting area 24 there is access, here a door 28, for each of the departments.

There is preferably additionally a pharmacy 30 within the building structure 10. The pharmacy 30 is preferably accessible from the reception area 22, or from an external pharmacy door 32. As such, registered patients of the integrated healthcare facility may access the pharmacy 30 directly from the reception area 22, whilst non-registered patients may access via the external door 32. The pharmacy 30 includes a counter 34 for pharmacy staff and for serving external and internal customers. The pharmacy 30 also includes a pharmacist's room 36.

Associated with the pharmacy is a waiting room 38 for patients who are waiting for the pharmacy is dispense prescription medication. The waiting room 38 includes chairs 39. Each of the general practitioner 12, optician 14, dentist 15, ENT specialist 16, and diagnostics centre 18 departments has individual rooms 40 or partitions where the patient would be assessed, examined and treated to maintain patient privacy and confidentiality. Additionally, each department may include a lavatory 42.

The building structure 10 may include a general lavatory 42, and a cafeteria 44 with a kitchen 46.

Referring now to FIG. 2, there is shown a second embodiment of an integrated healthcare facility of an integrated healthcare system. The facility includes a building structure 110 having at least one general practitioner 112, at least one optician 114, at least one dentist 115, at least one ear, nose and throat (ENT) specialist 116, and at least one diagnostics centre 118.

There is preferably a main access 120 to the building structure 110 which leads to a single reception area 122 and a single waiting area 124 for all the departments. The reception area 122 includes a reception staff area 126 and a management room 127. At the waiting area 124 there is access, here a door 128, for each of the departments.

There is preferably additionally a pharmacy 130 within the building structure 110. The pharmacy 130 is preferably accessible from the reception area 122, or from an external pharmacy door 132. As such, registered patients of the integrated healthcare facility may access the pharmacy 130 directly from the reception area 122, whilst non-registered patients may access via the external door 132. The pharmacy 130 includes a counter 134 for pharmacy staff and for serving external and internal customers. The pharmacy 130 also includes a pharmacist's room 136.

Associated with the pharmacy is a waiting room 138 for patients who are waiting for the pharmacy is dispense prescription medication. Patients who are waiting in the waiting room or elsewhere would be notified by the system when their prescription is ready.

The building structure 110 may include a lavatory area 142 which may have patient and staff lavatories. A cafeteria 144 may also be provided.

The integrated healthcare system further includes an integrated-healthcare-system server. The server may be at the facility within the building structure 10, remote from the building structure 10, or part within the building structure 10 and part remote from the building structure 10. The server may have a patient registration system including a plurality of different patient packages.

The patient packages may vary on cost. The cheapest package would be available for the lowest-risk and so healthiest patient. The packages may be ranked by the increasing cost. For example, there may be bronze, silver, gold, and platinum and diamond packages, in order of increasing cost. Alternatively, these may be termed the A, B, C, D, and E packages.

The packages as standard have access to the general practitioner 12. However, the patient may choose to have only access to general practitioner 12 as part of their package. Nevertheless, if the registered patient requires access for one-time appointment to any of optician 14, dentist 15, ENT specialist 16, and diagnostics centre 18, then the patient would pay a discounted fee, according to patient's contract. Alternatively, the packages can be customised to have access to any or all of the optician 14, dentist 15, ENT specialist 16, and diagnostics centre 18. Customizing the package for access to more departments results in higher cost.

Preferably, each department includes at least one video camera and at least one video display. The video camera and the video display are preferably communicated with the integrated-healthcare-system server.

The integrated healthcare system is preferably available for any person's use. However, unregistered patients may be required to make individual payments for any service, whilst registered patients may pay a regular fee for access to one or more departments, depending on their package.

To register, the prospective patient is on-boarded using an on-boarding method which may identify a healthcare package for the patient.

The method requires the prospective patient to have an electronic device with a processor, memory, and a display screen. The prospective patient may be required to confirm that the electronic device belongs to them.

The electronic device may, for example, be a smart mobile telephone or other portable electronic device. The electronic device may be configured to communicate with the integrated-healthcare-system server, for example via an internet connection.

The memory of the electronic device includes geographical history data. The geographical history data would preferably include the speed and duration of movement, as well the geographical position of the device. The speed, duration of movement, and geographical position of the device may preferably be measured by use of a global positioning system, cellular location system and/or an assisted global positioning system. Additionally or alternatively, the speed and duration of movement may be measured by an accelerometer.

The geographical history data is preferably at least in part based on a recorded duration or distance of movement of the electronic device in a given time period, wherein the movement has less than a given speed. For example, less than 9 metres per second [20.1 miles per hour], or less than 7 metres per second [15.7 miles per hour].

This may provide an approximate indication of the time or distance spent in exercise by the patient, for example running or cycling, in a given time period, since the speed limit may exclude time spent in motorised transport. The given time period may be a medium length of time, for example the preceding six months.

The geographical history data may also be assessed based on a predominate or most-common location of the electronic device. For example, the electronic device may record a most-common location of the electronic device. The memory of the electronic device or the integrated-healthcare-system server may have a location risk-factor correspondence data including a plurality of geographic locations and at least one health risk-factor associated with each location.

The electronic device also includes applications (apps) or computer programs installed on the electronic device. The applications may have a class or type. For example, the applications may have classes or types such as lifestyle, gaming, social media, news, and utility. The lifestyle class includes applications related to physical or mental health, fitness, and diet. The gaming class includes applications related to playing games. The social media class includes applications for social media platforms. The processor of the electronic device may be configured to monitor, and record on the memory, a duration of use of the applications in various classes.

Application data may be in the memory of the electronic device and may include the number of applications of a particular class type and/or the duration of use of applications of a particular class type. The application data may therefore include the number of lifestyle applications and the number of gaming applications and/or social media applications. The application data may also or alternatively include the duration of use of the lifestyle applications, and duration of use of gaming applications and/or social media applications.

To register via the on-boarding method, the patient may first be required to download and/or install an onboarding application (app), provided by the integrated healthcare system, onto the electronic device.

The patient may then input personal details into the application, such as their name, address, date of birth, and any national medical identifier that may be available. Such details may be communicated via the application to the integrated-healthcare-system server.

The onboarding application may then access the geographical history data and the application data of the electronic device. A first data value is then assigned to the geographical history data, as shown in step S101 in FIG. 3, and a second data value is assigned to the application data, as shown in step S102. This may be done at the electronic device.

The data values may be considered to be scores.

As such, the first data value may be assigned a high value if the geographical history data indicates large amounts of movement under the aforementioned speed limits, and/or a predominant location in an area having a low-risk factor. The first data value may be assigned a low value if the geographical history data indicates small amounts of movement and/or a predominant location in an area having a high-risk factor.

The second data value may be assigned a high value if the number of applications, and/or duration of use of applications, in the lifestyle class is high relative to the number of applications, and/or duration of use of applications, in the gaming and/or social media classes. The second data value may be assigned a low value if the number of applications, and/or duration of use of applications, in the lifestyle class is low relative to the number of applications, and/or duration of use of applications, in the gaming and/or social media classes.

The electronic device may then compute the first and second data values and determine whether a threshold level is reached, as per step S103. If the threshold level is not reached, then a first user health attribute may be applied. If the threshold level is reached or exceeded, then a second user health attribute may be applied. This is shown as per step S104.

As such, if the first user health attribute is applied, the patient may be considered to be relatively unhealthy, and therefore have a high associated risk-factor. If the second user health attribute is applied, the patient may be considered to be relatively healthy, and therefore have a low associated risk-factor.

It will be appreciated that there may be more than one threshold level, and therefore more than two health attributes may apply, allowing further gradation of health status and associated risk-factor. Most preferably, there are four thresholds, resulting in five health attributes corresponding to the bronze to diamond packages.

The first or second user health attributes are then outputted to the integrated-healthcare-system server, as per step S105 and S106 respectively.

The integrated-healthcare-system server may then determine proposed package data for the patient based on the health attribute. The proposed package data includes a determined package of any one of bronze to platinum, along with cost options for adding access to additional departments of optician 14, dentist 15, ENT 16, and diagnostics centre 18. The determined least healthy and highest risk patients are suggested the platinum package, and the most healthy and lowest risk patients are suggested the bronze package, with a graduated scale in between. The proposed package data may also include suggestions for those least healthy to select the option of access to the additional departments of optician 14, dentist 15, ENT 16, and diagnostics centre 18.

The integrated-healthcare-system server may then communicate the determined proposed package data to the electronic device, which may display this on the display screen, as per step S107.

The patient may then choose the suggested package, or another package, and choose whether they wish to have access to any or all of the additional departments of optician 14, dentist 15, ENT 16, and diagnostics centre 18, depending on their budget and personal circumstances.

The packages would provide the same level of care and/or service. However, patients determined for the Diamond package would require more frequency of visits and services, and the Bronze patient would require lesser frequency of visits and/or services. Accordingly, the Diamond patient would pay the higher payment and Bronze patient would pay lower payment. Therefore, the determined package would not affect the patient's level of care or service.

The least healthy patient would choose the platinum package out of necessity. Although a healthier patient may be suggested a lower rated package, such as a bronze package, a healthier patient may still decide to choose a higher rated package, such as the platinum package, for concern regarding their health in the future, or since they may underestimate their health.

The registration package may typically be offered for increments of one year. If the patient wishes to register for more than one year, then a discount may be applied.

The patient may then establish payment and/or pay an advance, and sign or agree to a contract. This may be done remotely, for example via the application on the electronic device, or physically, at the healthcare facility. A healthcare-system identity card may then be created and provided for the patient.

The patient is then registered, as per step S108.

Registration of a patient may provide the benefit of access to the general practitioner 12 department, and any other department if they selected the relevant customised package. The registered patient may also be offered a discount on prescribed pharmaceuticals and for one-time access to other departments which they may not have selected with their package.

However, registered patients may not be required to obtain prescribed pharmaceuticals from the pharmacy 30 at the integrated healthcare system, and they may decide to obtain pharmaceuticals from a third-party pharmacy. However, the registered patients would not obtain a discount from the third-party pharmacy and would not be able to claim from the integrated healthcare system.

The registered patient, regardless of package, may also be provided with an annual review to help identify and prevent life-threatening illnesses. Additionally, each package would provide one or more routine or general visits and one or more compulsory visit. In the instance that there are multiple integrated healthcare systems spread across a geographic area, the registered patient may be provided access to any of these with their identity card.

The registered patient may be able to book multiple appointments.

To book an appointment, a registered patient may provide to the integrated healthcare system the relevant facility name they wish to attend, the department they need to access, their symptoms, their practitioner gender preference, an urgency status, a preferred date and time, and whether the appointment should be in-person or via video or telephone. The appointment may then be confirmed by the system.

If the registered patient is accessing the general practitioner (12) department, or a department access to which they have added to their package, no payment is taken for the appointment, since the registered patient pays a recurring fee.

The registered patient may book an appointment via their electronic device which communicates with the integrated-healthcare-system server. This may be via a healthcare-system application installed on the electronic device. The healthcare-system application may be the same application as, or a different application to, the onboarding application.

Alternatively, the registered patient may book the appointment via a website, over the telephone, via email, or in person.

A non-registered patient may still book an appointment, and they may provide similar information, along with their personal identity information, although they will need to pay a fee.

A registered patient wishing to book an appointment at a department, access to which they have not added to their package, will need to pay a fee. However, the fee is reduced as compared to a non-registered patient.

The integrated healthcare facility may operate services out of hours for urgent medical matter, and this may only be for the registered patient. It will be appreciated that during that period of time, the facility may only operate via video and audio services.

Video appointments may be facilitated by the department video camera and the department video display, which are communicated via the integrated-healthcare-system server with a video camera and a video display on the electronic device. The healthcare-system application on the electronic device may provide the necessary interface for such video appointments.

Prior to an appointment, a patient may be reminded. This may be via various means, for example via the healthcare-system application on the electronic device, a message sent on a messaging platform, a text message sent via a mobile or cellular network, or email. The patient may choose the desired means and a frequency for the reminders.

The number of patients in a queue at the relevant department may be displayed to the patient. For example, this may be displayed as numerical information on the healthcare-system application on the electronic device. Alternatively, a video camera may record a physical queue at the integrated healthcare facility, and display this visually to the patient on the electronic device.

When a patient has an appointment at a department and is referred by a professional to have an appointment at a different department, the appointment at the different department may be scheduled immediately and on the same day. Therefore, the patient is easily able to access the referred appointment. Additionally, patient records can be shared between departments instantly.

Similarly, if a patient is prescribed a medicine, this prescription may be sent to the pharmacy 30 instantly. In particular, the healthcare professional would electronically prescribe one or more prescriptions, whether new or repeating, or a report, letter or similar. This would be sent from the relevant department 12, 14, 15, 16, 18 and would instantly go to the pharmacy 30, for example being displayed on a display screen. The prescription, report, or letter may also be displayed on the patient's electronic device.

The pharmacist would then make the appropriate preparations. Once the prescription is prepared, the patient would be notified, either via their electronic device, a display screen in the pharmacist waiting room 38, or via the pharmacist calling their name.

It is therefore possible to provide an integrated healthcare system having a plurality of different departments at a single site, thereby providing efficiency savings. Patients may register and pay a periodic fee so as to have access to the system, thereby allowing patients to access healthcare without large pay-as-you-go costs. Patients may be registered using data on their personal electronic device which allows for a relative risk-factor concerning their health to be determined. As such, patients may be offered more or less expensive periodic costs, depending on their anticipated use of the system given their health risk-factor. This allows for a fairer balance of patients across users, whilst still allowing for periodic charges, rather than pay-as-you-go charges.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. A healthcare-system on-boarding method of identifying a healthcare package for a patient to an integrated healthcare system by determining a general physical state of the patient, the method comprising:
at an electronic device with a processor, memory, and a display screen:
assigning a first data value to geographical history data in said memory;
assigning a second data value to application data installed on said electronic device;
based on the first and second data values, determining whether a threshold level is reached;
if the threshold level is not reached, applying a first user health attribute;
if the threshold level is at least any one of reached and exceeded, applying a second user health attribute;
outputting at least any one of the determined first and second user health attribute to an integrated-healthcare-system server;
receiving from the integrated-healthcare-system server a response based at least in part on at least any one of the outputted determined first and second user health attribute which comprises proposed package data; and
displaying the proposed package data on the display screen of the said electronic device.

2. The healthcare-system on-boarding method as claimed in claim 1, wherein the geographical history data is at least in part based on a duration of movement of the electronic device in a time period, wherein the movement has a speed of less than 9 metres per second [20.1 miles per hour].

3. The healthcare-system on-boarding method as claimed in claim wherein the geographical history data is at least in part based on a distance of movement of the electronic device in a time period, wherein the movement has a speed of less than 9 metres per second [20.1 miles per hour].

4. The healthcare-system on-boarding method as claimed in claim 1, wherein whether a threshold level is reached based on the geographical history data is at least in part based on a predominant location of the device.

5. The healthcare-system on-boarding method as claimed in claim 1, wherein at least any one of the memory and the integrated-healthcare-system server has location risk-factor correspondence data including a plurality of geographic locations and at least one health risk-factor associated with each location, whether a threshold level is reached based on the geographical history data is at least in part based on the corresponding health risk-factor associated with the location.

6. The healthcare-system on-boarding method as claimed in claim 1, wherein the application data is at least in part based on determining the number of applications installed on said electronic device in a first class and second class.

7. The healthcare-system on-boarding method as claimed in claim 1, wherein the application data is at least in part based on determining a duration of use of applications installed on said electronic device within a first class and second class.

8. The healthcare-system on-boarding method as claimed in claim 7, wherein the first class is lifestyle, and the second class is at least any one of gaming and social media.

9. he healthcare-system on-boarding method as claimed in claim 1, wherein the electronic device includes at least any one of a global positioning system, cellular location system and an assisted global positioning system which generates the geographical history data.

10. An integrated healthcare system using a healthcare-system on-boarding method as claimed in claim 1, the integrated healthcare system comprising:
a building structure at a single site;
a plurality of departments within the building structure, the plurality of departments including at least one general practitioner; at least one optician; at least one dentist; at least one ear, nose and throat specialist; at least one diagnostics centre, and at least one pharmacy;
an integrated-healthcare-system server in communication with the said electronic device;
a patient registration system on the said integrated-healthcare-system server, the patient registration system including a plurality of different patient packages;
one said patient package configured to be selected and sent to the said electronic device based at least in part on at least any one of the outputted determined first and second user health attribute.

11. The integrated healthcare system as claimed in claim 10, wherein the integrated-healthcare-system server includes a booking system configured to allow patients to book appointments with at least any one of the at least one general practitioner; the at least one optician; the at least one dentist; the at least one ear, nose and throat specialist; and the at least one diagnostics centre.

12. The integrated healthcare system as claimed in claim 11, wherein the booking system is configured to allow patients to book multiple appointments with at least any one of the at least one general practitioner; the at least one optician; the at least one dentist; the at least one ear, nose and throat specialist; and the at least one diagnostics centre.

13. The integrated healthcare system as claimed in claim 10, wherein the integrated-healthcare-system server is configured to send a reminder to the said electronic device for an appointment.

14. The integrated healthcare system as claimed in claim 10, further comprising at least one department video camera configured to image the department of at least any one of at least one general practitioner; at least one optician; at least one dentist; at least one ear, nose and throat specialist; and at least one diagnostics centre, the electronic device having a video display, the integrated-healthcare-system server configured to communicate with the electronic device so that the video display of the electronic device displays a video feed of the department video camera.

15. The integrated healthcare system as claimed in claim 10, further comprising at least one department video display at at least any one of the at least one general practitioner; at least one optician; at least one dentist; at least one ear, nose and throat specialist; and/or at least one diagnostics centre; the electronic device having a camera configured to image the patient, the integrated-healthcare-system server configured to communicate with the electronic device so that the department video display displays a video feed of the camera of the electronic device.

16. The integrated healthcare system as claimed in claim 10, wherein the patient package includes access to the general practitioner over a duration for a plurality of appointments at a fixed cost.

17. The integrated healthcare system as claimed in claim 10, wherein the package provides the option of an additional payment configured to allow access to at least any one of the optician; the ear, nose and throat specialist; and at least one diagnostics centre over a duration for a plurality of appointments at a fixed cost.

18. The integrated healthcare system as claimed in claim 16, wherein the duration is a year, access is configured to be a predetermined number of appointments dependent on the selected package, and the fixed cost is paid monthly.

19. The integrated healthcare system as claimed in claim 10, wherein the patient package provides a discount on at least any one of further medical products and further medical services.

20. The integrated healthcare system as claimed in claim 19, wherein at least any one of the further medical products and further medical services includes at least any one of medicines, consultations and treatments.
